(19) United States
(12) Patent Application Publication
(10) Pub. No.: US 2013/0022575 A1
(43) Pub. Date: Jan. 24, 2013

(54) SYSTEMS AND METHODS OF REPLACING INTESTINAL FLORA

(71) Applicant: Timothy Cassity, South Shore, KY (US)

(72) Inventor: Timothy Cassity, South Shore, KY (US)

(73) Assignee: MICROBIAL RX, Portsmouth, OH (US)

(21) Appl. No.: 13/186,274

(22) Filed: Jul. 19, 2011

(51) Int. Cl. A61K 35/74; C12N 1/20; A61P 1/00

(52) U.S. Cl. 424/93.3; 424/93.4; 435/252.4

(57) ABSTRACT

The invention relates to one or more systems and methods for creating a mixture of cultures used to replace intestinal flora. Specifically, the invention relates to methods and systems of treating diseases including Clostridium difficile and Crohn's disease by introducing a mixture of pure cultures of viable bacteria into the gastrointestinal tract.

### CROSS REFERENCE TO RELATED APPLICATION

This application claims the benefit of U.S. Provisional Application No. 61/356,777 filed, Jul. 20, 2010.

### BACKGROUND OF THE INVENTION

1. Field of Invention

The present invention generally relates to a system and method of using a mixture of cultures of bacteria to treat gastrointestinal diseases including Clostridium difficile associated diseases and Crohn's disease. More specifically, the present invention is in the technical field of using bacteria to replace flora lost due to antibacterial therapy.

2. Description of the Related Art

There are a plethora of diseases that may be remedied through the novel approaches disclosed by the present invention by means of replacing intestinal flora including but not limited to the treatment of Clostridium difficile and Crohn's disease.

Recurrent C. difficile infections are a growing burden and a Therapeutic challenge for patients and physicians. Current therapy consists of repeated courses of antibiotics with limited success rates and new therapeutic options are urgently needed. Fecal installations from healthy donors for the treatment of recurrent C. difficile infections seem a promising approach, restoring a normal bowel flora and preventing further outgrowth of C. difficile and its spores.

C. difficile is the most common infectious disease of the large intestine. C. difficile is the most common cause of antibiotic-associated diarrhea. C. difficile is the most common cause of nearly all cases of pseudomembranous colitis. The disruption of the normal flora of the large intestine is necessary before C. difficile can establish significant infection and produce toxins. In particular the destruction of the normal anaerobic flora permits the proliferation and toxin production by toxigenic strains of C. difficile. Antibiotic use is associated with C. difficile associated disease in that it disrupts the normal bacteria flora of the large intestine. A wide variety of antibiotics can predispose a patient to C. difficile Associated Disease (CDAD).

The disease appears most often when patients are treated with antibiotics that are excreted through the large intestine and kill Bacteroides species and other anaerobic flora of the large intestine. C. difficile is the consequence of the production of toxins A & B, which may proliferate and produce toxins when not held in check by normal bacterial flora of the large intestine.

Nearly all reported patients with recurrent C. difficile are treated with donor faeces (or stool transplant) are cured after one or more infusions. Clinical improvement can be noticed within a few days following donor faeces infusion. Many patients report follow-up of less than one month, which implies that definite success rates. The invention disclosed herein will be at least as effective as treating with fecal infusion and will provide benefit by having an ensured quality not found when accepting stool from donors and will additionally escape the negative feelings that often associate receiving a stool transplant.

CDAD are the most frequently identified causes of nosocomial diarrhea producing both endemic and epidemic diarrhea. CDAD incidence ranges from <1% to 7.8% of hospital discharges. CDAD prolongs hospitalization, increases the costs of care, and causes considerable morbidity, especially in the 10-20% who relapse, and a mortality of 0.6-2.3%. Elderly patients and those who have long hospital stays are at particularly high risk. Horizontal transmission of C. difficile in the hospital to susceptible patients receiving antibiotics accounts for the vast majority of CDAD incidence. No infection control method has been widely successful at preventing transmission, and prophylactic measures aimed at preventing symptoms if transmission occurs have proven cumbersome, ineffective, or both. Therefore, new and innovative approaches to prevent CDAD are needed.

C. difficile related diseases also cause significant damages in animals. For instance, in horse breeding, young foals are extremely susceptible. In young foals, infection generally results in death. Antibiotic treatment in animals is not only expensive, but is not completely effective. The embodiments of the disclosed invention provide a lower cost and more effective solution to treatment of clostridium difficile related diseases.

Patients with a first C. difficile infection typically respond to metronidazole or oral vancomycin. However, patients with recurrent C. difficile Infections tend not to respond to either of these treatments. In the current state of the art, treatments include prolonged oral vancomycin pulse and or tapering schedules. However, there are no treatments that have worked consistently. The role of immunoglobulins, whey prepared from vaccinated cows, probiotics or other antibiotics is also unproven. One treatment that has had some level of success is a treatment strategy where fecal infusions are successfully given for the treatment of recurrent C. difficile infections. Restoring intestinal flora has been historically thought of as the mechanism responsible for successfully treating patients with recurrent C. difficile infections. More than 150 patients thus far have successfully received feces from a healthy donor, either infused through an enema, or through a nasoduodenal or nasogastric tube.

Due to a lack of clinical trials, fecal installations often are offered only to patients with more than two relapses, since it is still considered a last, uncommon, and rather distasteful rescue therapy. Currently, adult patients with proven recurrent C. difficile infections can be included in the first randomized controlled study comparing donor faeces installation with antibiotic therapy.

One drawback to current fecal transplants includes the necessity of donor screening. Early reports on fecal installation only mention that donors who had used antibiotics in the preceding months were excluded. Although transmission of infectious diseases has not been reported after fecal infusions, most publications from the past decade report extensive screening of donors. Most donors are sought in relative proximity of the patient (partners, relatives, household members). However, there is no rationale to exclude healthy volunteers. Many reports fail to mention the exact origin of the donors and an investigation of patient preferences is lacking. We do not apply any restrictions concerning the food intake of donors prior to donation. Although there can be potential important differences in the quality of the microbiota present in donor faeces from different individuals, historically their intestinal flora has not been analyzed prior to use for fecal infusion. Information is lacking with regard to the specific groups and amount of bacteria necessary for optimal restoration of intestinal flora, thereby preventing C. difficile to become clinically significant.

Typically, infusing faeces through colonoscopy is more difficult and strenuous, whereas infusion through a nasoduodenal tube seems safe and time-efficient. A disadvantage of a nasoduodenal/jejunal tube is that donor feces may be difficult to install if patients have signs of diminished passage of fluids through their intestines. On the other hand, infusing feces using this route has the advantage that the infused flora reaches the whole bowel.

### BRIEF DESCRIPTION OF EXEMPLARY EMBODIMENTS

The present invention generally relates to a system and method for using a mixture of cultures of bacteria commonly found in the human large intestine and can be used to replace flora lost due to antibacterial therapy. Exemplary embodiments of the present are an improvement on current treatments for gastrointestinal disease. Prior to the disclosure of the exemplary embodiments, the most successful treatment of CDAD and Crohn's disease has been a fecal transplant therapy. One of ordinary skill in the art will readily recognize that the current invention is a notable improvement upon fecal transplant therapy because it offers a cost-effective, highly successful method of treating gastrointestinal problems while decreasing the risk of infection and eliminating the need for donor screening.

Flora of the normal large intestine typically consists of Bacteroides species, particularly those of the Bacteroides fragilis group, at a concentration of 10̂11 per gram of feces. Bacteroides thetaiotaomicron is the most prevalent of the bacteroides species found in the large intestine. Other anaerobic organisms normally present in the large intestine include Bifidobacterium species. and Eubacterium species. Eubacterium spp. are found at a concentration of 10̂10 in most stools as well. The most common species are Eubacterium aerofasciens, Eubacterium cylindroides, Eubacterium lentum, and Eubacterium rectale. The most common Bifidobacterium species are Bifidobacterium bifidum and Bifidobacterium longu. Facultative organisms predominantly include Escherichia coli and Enterococcus species. Of the Enterococcus spp, Enterococcus faecalis is the most common. E. coli is also found in normal stool flora, but usually represents about 1% of the total bowel flora.

Further embodiments of the present invention, as well as the structure and operation of these embodiments of the present invention, are described in detail below with reference to the accompanying drawings.

While the specification concludes with claims particularly pointing out and distinctly claiming the invention, it is believed that the embodiments will be better understood from the following description in conjunction with the accompanying FIG.s, in which like reference numerals identify like elements and in which:

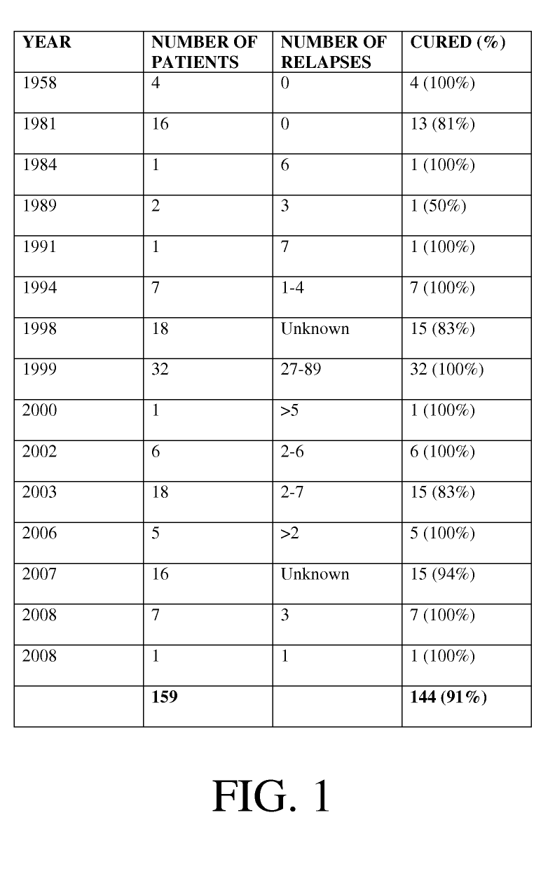

FIG. 1 provides a chart which provides a history of fecal therapy for recurrent C. difficile infections;

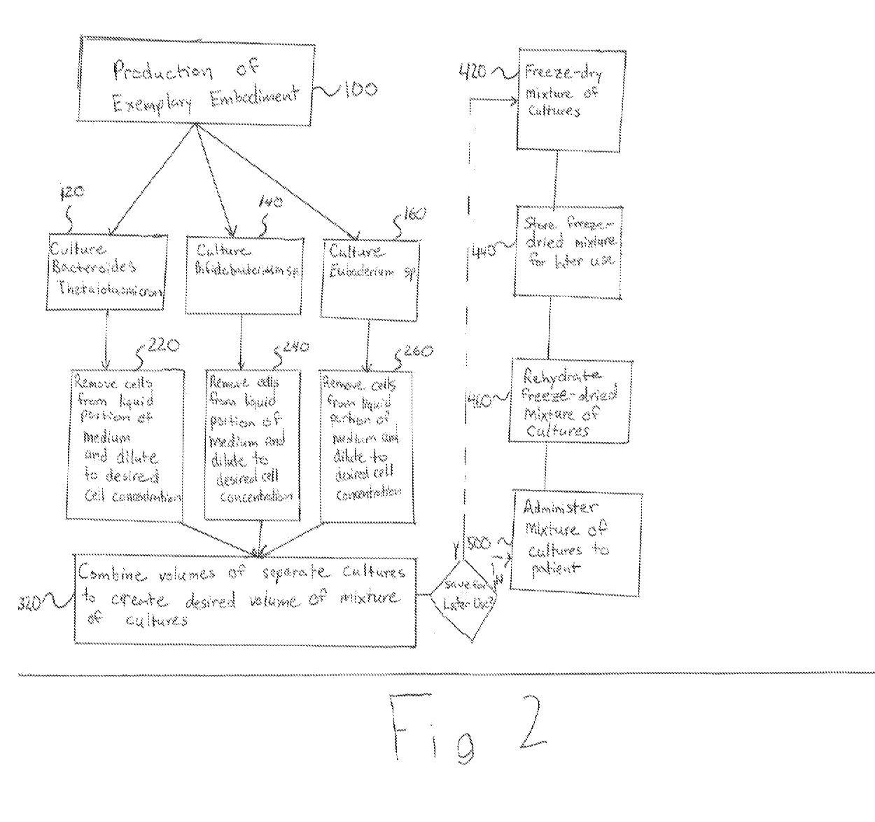

FIG. 2 provides a flowchart describing the production of an exemplary embodiment of the disclosed invention;

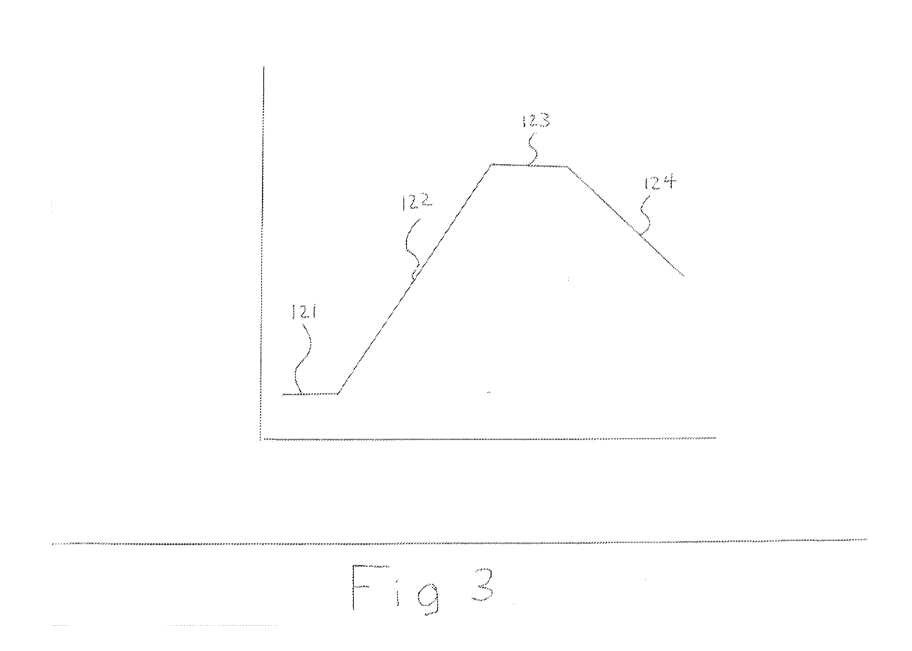

FIG. 3 provides a growth rate chart showing how long the bacteria should be grown in an exemplary embodiment;

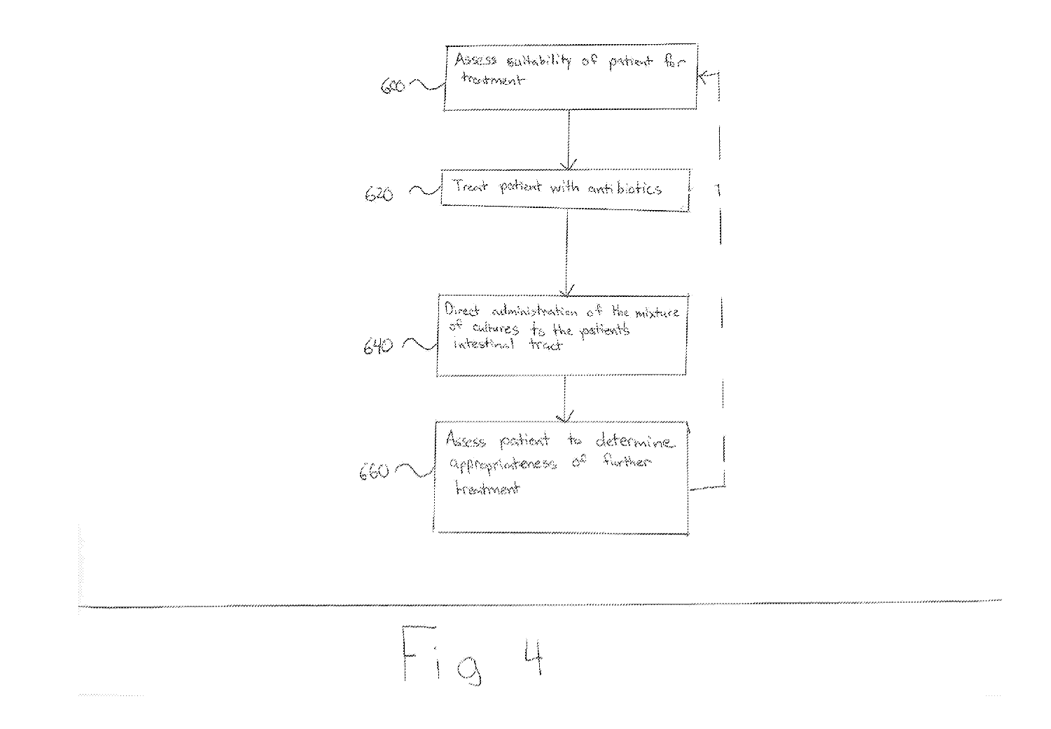

FIG. 4 provides an exemplary embodiment of the administration of the mixture of cultures to a patient.

### DETAILED DESCRIPTION OF EXEMPLARY EMBODIMENTS

The system and method described herein provides a solution for solving various gastrointestinal problems by replacing flora lost due to antibacterial therapy. As previously described, the most successful treatment for CDAD has been via fecal transplant therapy. Exemplary embodiments of the present invention are anticipated to be at least as effective as fecal transplant therapy in the treatment of CDAD.

FIG. 1 provides a chart which provides a history of fecal therapy for recurrent C. difficile infections. As is shown by the chart, fecal therapy has been quite successful in the treatment of C. difficile infections whereas other treatments have failed to be consistently effective. In fact, of the 159 patients represented in FIG. 1, 91% of patients with recurrent C. difficile treated with donor faeces were cured after one or more infusions. In an exemplary embodiment, the mixture of bacteria mimics normal intestinal flora such as those used when treated with donor infusions so it is foreseeable that the mixture of cultures found in the exemplary embodiments herein would have a success rate greater than or equal to treatment using donor faeces. It is anticipated that the exemplary embodiments presently disclosed may be administered to patients through a variety of methods. Methods for administration of exemplary embodiments of the invention include those methods used in administering donor faeces in Table 1. Of reported patients receiving donor faeces, 80% were given a fecal installation through enema or colonoscope, and 20% received the faeces through a nasogastric or nasoduodenal/jejunal tube.

FIG. 2 provides an exemplary embodiment of the production of the mixture of cultures used to replace intestinal flora. In the exemplary embodiment, the substance used to replace intestinal flora is a mixture including Bacteroides thetaiotaomicron, Eubacterium spp., and Bifidobacterium spp. As shown in FIG. 2, the exemplary embodiment is cultured and grown in a typical manner for growing bacteria. In an exemplary embodiment, the three organisms being B. thetaiotaomicron in Step 120, Bifidobacterium spp. in Step 140, and Eubacterium spp. in Step 160, are cultured separately because they each grow at a different rate. In the exemplary embodiment of FIG. 2, the selected organisms are cultured separately in steps 120, 140, and 160 in a chopped meat broth. Any other medium suitable for growing bacteria may be used in place of chopped meat broth. It is anticipated that certain types of selective media may present advantages in growing embodiments of the current invention because they would allow bacteroides to grow more quickly.

In the exemplary embodiment of FIG. 3, the cultures are grown at 37 C for 48-72 hours under anaerobic conditions. FIG. 3 provides a standard bacterial growth curve outlining the phases of growth for bacteria. As shown in FIG. 3, bacteria undergo a lag phase 121, an exponential growth phase 122, a stationary phase 123, and a death phase 124. To maximize the shelf life of the mixture of cultures the cells should be removed from the liquid portion of the medium as shown in Steps 220, 240 and 260 of FIG. 2, during the logarithmic phase of the bacterial growth curve represented by phase 122 in FIG. 3. For the bacteria selected in FIG. 2 of the exemplary embodiment, the exponential phase 122 is estimated to occur when the bacteria have been growing at 37 C for between 48-72 hours.

The mixture of cultures may be grown in small batches or growth conditions can be adapted and optimized for commercial-size fermentation processes for production on a large scale.

An active bacterium in an exemplary embodiment, is Bacteroides found in Step 120 and 220 of FIG. 2. Bacteroides are normally mutualists, making up the most substantial portion of the mammalian gastrointestinal flora, where they play a fundamental role in processing of complex molecules to simpler ones in the host intestine. As may as 10̂10-10̂11 cells per gram of human feces may be present in healthy stool. In an exemplary embodiment, 10̂10-10̂11 cells per gram of human feces are present to mimic normal stool. They can use simple sugars when available but the main source of energy is polysaccharides from plant sources.

Bacteroides spp. benefit their host by excluding potential pathogens from colonizing the gut. Some species are opportunistic human pathogens, causing infections of the peritoneal cavity, gastrointestinal surgery, and appendicitis via abscess formation, inhibit phaycytosis, and inactivating beta-lactam antibiotics.

Bacteroides are resistant to a wide variety of antibiotics and recently many species have acquired resistance to erythromycin and tetracycline. This high level of antibiotic resistance has prompted concerns that Bacteroides spp. may become a reservoir for resistance in other, more highly-pathogenic bacterial strains.

One intended use of the present invention is for therapy for recurrent CDAD. An exemplary embodiment may be used as a defined replacement for donor stool in stool transplant on stool instillation procedures.

B. thetaiotaomicron is the fastest growing anaerobic bacteria in natural feces and is the active ingredient in the exemplary embodiment. B. thetaiotaomicron is an attractive option because it is fast growing and effective. However, it is anticipated that other species of Bacteroides are also suitable for use in embodiments of the invention. Bacteroides spp. that are expected to work in the present invention include but are not limited to B. fragilis, B. vulgatis, and B. distatonis. Alternatively, a mixture of Bacteroides spp. may be effective in exemplary embodiments of the present invention. In addition to or in place of B. thetaiotaomicron, other species of Bacteroides may be used. It is anticipated that the disclosed method is successful using B. fragilis, B. vulgatis, B. distatonis or B. thetaiotaomicron either independently or in combination with other species of Bacteroides. The higher concentration of bacteroides placed into the gastrointestinal system the more effective for overrunning the C. difficile.

Eubacterium spp. in Step 160 and Bifidobacterium spp. in Step 140 further comprise the exemplary embodiment of FIG. 2. Eubacterium spp. and Bifidobacterium spp. are not necessary to the success of the mixture yet their inclusion may be advantageous because they are commonly found in normal stool.

The most common species of Eubacterium found in stool and which may be selected in exemplary embodiments of the present invention are E. aerofasciens, E. cylindroides, E. lentum and E. rectale. The most common species of Bifidobacterium found in stool are B. bifidum and B. longum.

Eubacterium spp. found in Step 160 and Step 260 and Bifidobacterium spp. found in Step 140 and 240 of the exemplary embodiment in FIG. 2 mimic anaerobic flora of normal stool and if a patient receiving the mixture of cultures found in the exemplary embodiment is still getting antibiotics or has effects from antibiotics, Eubacterium spp. and Bifidobacterium spp. may be resistant to the passive bacteria found in stool.

In the exemplary embodiment of FIG. 2, each organism in the mixture is cultured separately as shown in Steps 120, 140, and 160, because the bacteria have different growth rates. After the organisms are individually cultured in Steps 120-160 in the exemplary embodiment of FIG. 2, the cells from each cultured organism are removed from the liquid portion of the medium and diluted to a desired cell concentration in Steps 220, 240, and 260. In an exemplary embodiment, the cultured organisms may be diluted with saline or water.

In one embodiment of FIG. 2, equal quantities of bacterium with McFarland solution of three or greater are combined in Step 320. The volumes of separate cultures may be mixed to achieve various concentrations of cultures which may be advantageous for the replacement of intestinal flora. In Step 320 of the exemplary embodiment of FIG. 2, the mixture is comprised of a 300 mL infusion in a chopped meat broth containing approximately: 1×10̂10 B. thetaiotaomicron, 1×10̂9 E. cylindroides, 1×10̂9 B. bifidum.

Once the mixture of cultures has been prepared in Step 320 of the exemplary embodiment of FIG. 2, the mixture may be administered to a patient in Step 500 or it may be freeze-dried in Step 420 and stored in Step 440 for later use. It is anticipated that exemplary embodiments of the mixture of cultures may remain viable through a process of freeze-drying and rehydrating. Freeze-drying the mixture of cultures may be advantageous because it is anticipated that the most effective embodiments of the mixture of cultures is a moist mixture. In an embodiment where the mixture of cultures has been freeze-dried, it may be rehydrated as shown instep 460 of the exemplary embodiment of FIG. 2. Among other methods of rehydration, the mixture of cultures may be rehydrated just prior to use with 0.9% sodium chloride solution. Following preparation of the mixture of cultures in Step 320, or following rehydration of freeze-dried mixture in Step 460 of FIG. 2, the mixture of cultures may be administered to a patient in Step 500 of the exemplary embodiment to replace intestinal flora.

FIG. 4 provides an exemplary administration of the mixture of cultures to a patient and a method of treating a patient to replace the patient's intestinal flora. Prior to administration, patients should be assessed as to whether they are suitable candidates for receiving administration of the mixture of cultures for replacement of intestinal flora as shown in Step 600 of FIG. 4. The mixture of cultures may not be administered to patients with pre-existing pseudomembranous colitis or those critically ill with Clostridium difficile associated disease with the intended consequences.

In the exemplary embodiment of FIG. 4, a patient receiving the mixture of cultures disclosed by one or more of the exemplary embodiments disclosed herein is treated with oral vancomycin prior to administration of the mixture of cultures as shown in Step 620. While it is not necessary to pretreat with oral vancomycin, it is anticipated that there are advantages to pretreating with an antibiotic prior to administration of the mixture of cultures to a patient. It is further anticipated that Step 620 is not necessary to the usefulness of the invention in some embodiments. When treated a CDAD, oral vancomycin may be used to kill as many viable C. difficile vegetative cells as possible prior to replacing intestinal flora.

As discussed above, the mixture of cultures disclosed in the exemplary embodiments herein may be administered to patients through a variety of means including those which have previously been used to patients receiving donor faeces. In the exemplary embodiments disclosed in FIG. 4, the mixture of cultures is administered directly into the gastrointestinal system via a nasoduodenal tube, nasogastric tube, or by enema during Step 640. The nasoduodenal or nasogastric tube may be advantages because the entire large intestine is exposed to the mixture of cultures. A possible disadvantage of a nasoduodenal tube is that donor faeces may be difficult to install if patients have signs of diminished passage of fluids through their intestines. If using a nasoduodenal or nasogastric tube, a proton pump inhibitor may be advisable to be given prior to instillation of the mixture of cultures. It is advantageous to administer the mixture of cultures directly to a patient's intestinal tract rather than introducing the mixture of cultures through an IV because IV administration may result in profuse bacteremia.

It is anticipated that some patients with recurrent C. difficile infections may be cured after one infusion of the mixture of cultures disclosed in exemplary embodiments herein. However, some patients may benefit from multiple administrations of the mixture of cultures disclosed in the exemplary embodiments. Therefore, patients receiving an administration of the mixture of cultures must be monitored and assessed whether they would benefit from multiple administrations of the mixture of cultures as shown in Step 660 of FIG. 4. There are no known side effects to the mixture of cultures and consequently patients will not suffer from multiple administration of the mixture of cultures.

In addition to treating CDAD, it is anticipated that the disclosed invention will be useful in treating other problems associated with the intestine including Crohn's disease.

Treatment of Crohn's disease using one or more described embodiments of the invention involves the same treatment as used when treating CDAD which is disclosed herein.

All documents cited are, in relevant part, incorporated herein by reference; the citation of any document is not to be construed as an admission that it is prior art with respect to the present invention.

While various embodiments of the present invention have been described above, it should be understood that they have been presented by way of example, and not limitation. It will be apparent to persons skilled in the relevant art(s) that various changes in form and detail can be made therein without departing from the spirit and scope of embodiments of the present invention. Thus, embodiments of the present invention should not be limited by any of the above described exemplary embodiments, but should be defined only in accordance with the following claims and their equivalents.

In addition, it should be understood that the figures in the attachments, which highlight the structure, methodology, functionality and advantages of embodiments of the present invention, are presented for exemplary purposes only. Embodiments of the present invention are sufficiently flexible and configurable, such that it may be implemented in ways other than that shown in the accompanying figures.

Further, the purpose of the foregoing Abstract is to enable the U.S. Patent and Trademark Office and the public generally, and especially the scientists, engineers and practitioners in the relevant art(s) who are not familiar with patent or legal terms or phraseology, to determine quickly from a cursory inspection the nature and essence of this technical disclosure. The Abstract is not intended to be limiting as to the scope of embodiments of the present invention in any way.

What is claimed is:

1. A system of replacing intestinal flora comprising growing a mixture of cultures which includes one or more species of Bacteroides and administering said mixture into a gastrointestinal system.

2. The system of claim 1 including Bacteroides thetaiotaomicron.

3. The system of claim 1 including Bacteroides fragilis.

4. The system of claim 1 including Bacteroides vulgatis.

5. The system of claim 1 including Bacteroides distatonis.

6. The system of claim 1 wherein the mixture of cultures further comprises Eubacterium cylindroides.

7. The system of claim 1 wherein the mixture of cultures further comprises Bifidobacterium bifidum.

8. The system of claim 1 wherein the mixture of cultures comprises a 300 ml infusion containing 1×10̂10 Bacteroides thetaiotaomicron, 1×10̂9 Eubacterium cylindroides, and 1×10̂9 Bifidobacterium bifidum.

9. The system of claim 1 wherein the mixture of cultures is administered via a nasoduodenal tube into a gastrointestinal system.

10. The system of claim 1 wherein the mixture of cultures is administered via a nasogastric tube into a gastrointestinal system.

11. The system of claim 1 wherein the mixture of cultures is administered via an enema into a gastrointestinal system.

12. A method of producing cultures used to restore intestinal flora comprising:
a. creating a mixture of cultures including Bacteroides thetaiotaomicron, Eubacterium cylindroides, and Bifidobacterium bifidum wherein each organism is cultured separately;
b. growing the cultures under anaerobic conditions;
c. After incubating the cultures, removing the liquid portion of the medium and diluting the culture with a medium suitable for growing cultures to create a final preparation.

13. The method of claim 12 wherein the mixture of cultures are grown for 48-72 hours.

14. The method of claim 12 wherein the final preparation is freeze-dried.

15. The method of claim 14 wherein the final preparation is rehydrated.

16. The method of claim 15 wherein the final preparation is rehydrated using a sodium chloride solution.
